(19) United States
(12) Patent Application Publication
(10) Pub. No.: US 2023/0212104 A1
(43) Pub. Date: Jul. 6, 2023

(54) FORMING ACETIC ACID BY THE SELECTIVE OXIDATION OF LIGHT HYDROCARBONS

(71) Applicant: NOVA CHEMICALS (INTERNATIONAL) S.A., Fribourg (CH)

(72) Inventors: Shahin GOODARZNIA, Calgary (CA); Bolaji OLAYIWOLA, Calgary (CA); Vasily SIMANZHENKOV, Calgary (CA)

(21) Appl. No.: 18/008,782

(22) PCT Filed: May 25, 2021
(86) PCT No.: PCT/IB2021/054550

§ 371 Date: Dec. 7, 2022

Related U.S. Application Data
(60) Provisional application No. 63/036,482, filed on Jun. 9, 2020.

(51) Int. Cl. C07C 51/15; B01J 19/24; B01J 23/22; B01J 23/00

(52) U.S. Cl. CPC C07C 51/15; B01J 19/2445; B01J 23/002; B01J 23/22; B01J 2523/55; B01J 2523/56; B01J 2523/64; B01J 2523/68; B01J 2523/824

(57) ABSTRACT

Methods and a reactor system for producing acetic acid in a selective oxidation (SO) reactor are provided. An example method includes providing a fresh feed stream to the SO reactor, wherein the fresh feed stream includes a light hydrocarbon feed stream, a carbon dioxide feed stream, and a steam feed stream. Acetic acid is formed in the SO reactor. An acetic acid product stream is separated from a reactor effluent stream in a scrubber. A recycle gas stream is obtained from the scrubber. At least a portion of the recycle gas stream is combined into the fresh feed stream to the SO reactor.

### TECHNICAL FIELD

The present disclosure relates generally to selective oxidation (SO) of lower alkanes to form acetic acid in large amounts.

### BACKGROUND ART

Olefins like ethylene, propylene, and butylene, are basic building blocks for a variety of commercially valuable polymers. Since naturally occurring sources of olefins do not exist in commercial quantities, polymer producers rely on methods for converting the more abundant lower alkanes into olefins. The method of choice for today’s commercial scale producers is steam cracking, a highly endothermic process where steam-diluted alkanes are subjected very briefly to a temperature of at least 800° C. The fuel demand to produce the required temperatures and the need for equipment that can withstand that temperature add significantly to the overall cost. Also, the high temperature promotes the formation of coke which accumulates within the system, resulting in the need for costly periodic reactor shutdown for maintenance and coke removal.

Selective oxidation processes, such as oxidative dehydrogenation (ODH), are an alternative to steam cracking that are exothermic and produce little or no coke. In ODH, a lower alkane, such as ethane, is mixed with oxygen in the presence of a catalyst and optionally an inert diluent, such as carbon dioxide or nitrogen or steam, in some embodiments at temperatures as low as 300° C., to produce the corresponding alkene. In some embodiments, various other oxidation products may also be produced in this process.

### SUMMARY OF INVENTION

An embodiment described herein provides a method for producing acetic acid in a selective oxidation (SO) reactor. The method includes providing a fresh feed stream to the SO reactor. The fresh feed stream includes a light hydrocarbon feed stream, a carbon dioxide feed stream, and a steam feed stream. Acetic acid is formed in the SO reactor. An acetic acid product stream is separated from a reactor effluent stream in a scrubber. A recycle gas stream is obtained from the scrubber, and at least a portion of the recycle gas stream is combined into the fresh feed stream to the SO reactor.

Another embodiment described herein provides a reactor system for producing acetic acid in a selective oxidation process. The reactor system includes a selective oxidation (SO) reactor. The SO reactor includes a number of feed lines including a light hydrocarbon feed line, a carbon dioxide feedline, and a steam feedline. The SO reactor includes an SO catalyst to convert feedstocks, at least in part, to acetic acid, and a reactor effluent line. The reactor system includes a scrubber coupled to the reactor effluent line. The scrubber includes an acetic acid product line and a separated gas outlet. The separated gas outlet is coupled to one of the number of feed lines to recycle at least a portion of a gas stream separated from an acetic acid product stream to the SO reactor.

Another embodiment described herein provides a method for producing acetic acid in a selective oxygenation (SO) reactor. The method includes providing a fresh feed stream to the SO reactor. The fresh feed stream includes an ethane feed stream, a carbon dioxide feed stream provided from flue gas stream from a combustion process, and a steam feed stream. Acetic acid is formed in the SO reactor, wherein an amount of water added to the SO reactor as the steam feed stream is adjusted to increase selectivity for acetic acid. An acetic acid product stream is separated from a reactor effluent stream in a scrubber. A recycle ethane stream is obtained from the scrubber, and at least a portion of the recycle ethane stream is combined into the fresh feed stream to the SO reactor.

The details of one or more implementations are set forth in the accompanying drawings and the description below. Other features and advantages will be apparent from the description and drawings, and from the claims.

### BRIEF DESCRIPTION OF DRAWINGS

FIG. 1 is a block diagram of a selective oxidation system for the production of acetic acid from the selective oxidation of light hydrocarbons in the presence of water, in accordance with examples.

FIG. 2 is a block diagram of a chemical complex that integrates a selective oxidation (SO) system into the chemical complex for the production of acetic acid from the selective oxidation of ethane and ethylene in the presence of water, in accordance with examples.

FIG. 3 is a block diagram of a selective oxidation system for the production of acetic acid from the selective oxidation of a mixture of ethane and ethylene in the presence of water, in accordance with examples.

FIG. 4 is a simplified process flow diagram of a chemical complex used for the production of ethylene in an oxidative dehydrogenation (ODH) reaction, in accordance with examples.

FIG. 5 is a simplified process flow diagram of an SO reactor system for the production of acetic acid, in accordance with examples.

FIG. 6 is a simplified process flow diagram for using a group of parallel SO reactors wherein one SO reactor is used in a swing capacity to produce acetic acid, while other reactors are producing ethylene, in accordance with examples.

FIG. 7 is a simplified process flow diagram of a reactor system for using a group of parallel SO reactors wherein one SO reactor is used in a swing capacity to produce acetic acid, while other SO reactors are producing ethylene, in accordance with examples.

FIG. 8 is a simplified process flow diagram of a process skid for the production of acetic acid from a light hydrocarbon and carbon dioxide, in accordance with examples.

FIG. 9 is a block flow diagram of a method for the production of acetic acid in a selective oxidation reactor, in accordance with examples.

FIG. 10 is a drawing of a reactor used for simulation.

### DESCRIPTION OF EMBODIMENTS

Selective oxidation (SO) is generally used in ODH reactions to form ethylene, alpha-olefins, other olefins, and dienes from ethane. Examples disclosed herein describe the use of SO to convert carbon dioxide and light hydrocarbons, such as ethane or mixtures of ethane and ethylene, into acetic acid in the presence of steam. An SO catalyst, such as an ODH catalyst, may be used to provide very high selectivity for the formation of acetic acid using conventional ODH operating conditions, for example, at an operating temperature of about 250° C. to about 450° C., a reactor inlet pressure of about 5 psig to about 75 psig, a GHSV of about 200 hr⁻¹ to about 10000 hr⁻¹, a WHSV of about 0.2 hr⁻¹ to about 10 hr⁻¹, and a feed linear velocity of greater than about 5 cm/sec. A stand-alone SO reactor may be used, for example, on a skid-mount. In some examples, an SO reactor in an ODH complex may be used in a swing capacity, allowing other reactors to continue to produce ethylene or other products. The feed can be provided from a steam cracker or other units in an ODH complex. In some cases, at least a portion of the feed can be provided as flue gas from power plants or other combustion processes, providing a path for sequestration of CO₂ emissions.

The process may be used in a standalone configuration, for example, using a process skid, or may be used in a reactor in an ODH complex. In the process, acetic acid is efficiently generated from ethane using CO₂ as an oxidizing agent. The acetic acid may then be sold or may be converted to other products. Further, the techniques may reduce CO₂ emission by converting it into acetic acid.

The techniques may be integrated into an ODH complex, providing additional control over the process by sending a portion (or all) of the unreacted ethane and generated CO₂ in an ODH reactor to an SO reactor for the generation of acetic acid, then a portion of the generated acetic acid back to the ODH reactor to suppress or reduce acetic acid formation and, thus, enhance ethylene formation in this reactor. The other portion of acetic acid can be sold or be converted to other value products.

Alternately, during times of low ethylene demand, ethane can be used in an SO reactor to produce acetic acid, by switching the feed to this reactor from ethane/O₂/diluent to ethane/CO₂/steam. The use of the CO₂ may reduce emissions by converting the CO₂ to acetic acid. For both cases, the generated acetic acid can optionally be converted to other value products such as ethanol or ethylene based on market need.

The process is not limited to the use of ethane as a feed to an SO reactor, but may use a mixture of ethylene and ethane, for example, converting a product stream from an ODH reactor to acetic acid. The product stream from the ODH reactor may include ethane, ethylene, and carbon dioxide, which, with added steam, may be converted into oxygenated compounds, such as acetic acid, with nearly 100% selectivity. Alternatively, this process can be used integrated with a conventional steam cracking or ODH process.

Other than in the operating examples or where otherwise indicated, all numbers or expressions referring to quantities of ingredients, reaction conditions, etc. used in the specification and claims are to be understood as modified in all instances by the term “about”. Accordingly, unless indicated to the contrary, the numerical parameters set forth in the following specification and attached claims are approximations that can vary depending upon the desired properties, which the present disclosure desires to obtain. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the disclosure are approximations, the numerical values set forth in the specific examples are reported as precisely as possible. Any numerical values, however, inherently contain certain errors necessarily resulting from the standard deviation found in their respective testing measurements.

Also, it should be understood that any numerical range recited herein is intended to include all sub-ranges subsumed therein. For example, a range of “1 to 10” is intended to include all sub-ranges between and including the recited minimum value of 1 and the recited maximum value of 10; that is, having a minimum value equal to or greater than 1 and a maximum value of equal to or less than 10. Because the disclosed numerical ranges are continuous, they include every value between the minimum and maximum values. Unless expressly indicated otherwise, the various numerical ranges specified in this application are approximations.

As used herein, the term “alkane” refers to an acyclic saturated hydrocarbon. In many cases, an alkane consists of hydrogen and carbon atoms arranged in a linear structure in which all of the carbon-carbon bonds are single bonds. Alkanes have the general chemical formula CₙH₂ₙ₊₂. In many embodiments of the disclosure, alkane refers to one or more of methane, ethane, propane, butane, pentane, hexane, octane, decane and dodecane. In particular embodiments, alkane refers to ethane and propane.

As used herein, the term “alkene” refers to unsaturated hydrocarbons that contain at least one carbon-carbon double bond. In many embodiments, alkene refers to alpha olefins. In many embodiments of the disclosure, alkene refers to one or more of ethylene, propylene, 1-butene, pentene, pentadiene, hexene, octene, decene and dodecene. Further, as used herein, the term includes other compounds with carbon-carbon double bonds, such as butadiene, among others. In particular embodiments, alkene refers to ethylene and propylene and, in some embodiments, ethylene.

As used herein, the terms “alpha olefin” or “α-olefin” refer to a family of organic compounds which are alkenes (also known as olefins) with a chemical formula CₓH₂ₓ, distinguished by having a double bond at the primary or alpha (α) position. In many embodiments of the disclosure, alpha olefin refers to one or more of ethylene, propylene, 1-butene, 1-pentene, 1-hexene, 1-octene, 1-decene and 1-dodecene. In particular embodiments, alpha olefins refer to ethylene and propylene and, in some embodiments, ethylene.

As used herein, the term “essentially free of oxygen” means the amount of oxygen present, if any, remaining in a process stream after the one or more ODH reactors, and in many embodiments after the second reactor as described herein, is low enough that it will not present a flammability or explosive risk to the downstream process streams or equipment.

As used herein, the term “fixed bed reactor” refers to one or more reactors, in series or parallel, often including a cylindrical tube filled with catalyst pellets with reactants flowing through the bed and being converted into products. The catalyst in the reactor may have multiple configurations including, but not limited to, one large bed, several horizontal beds, several parallel packed tubes, and multiple beds in their own shells.

As used herein, the term “fluidized bed reactor” refers to one or more reactors, in series or parallel, often including a fluid (gas or liquid) which is passed through a solid granular catalyst, which can be shaped as tiny spheres, at high enough velocities to suspend the solid and cause it to behave as though it were a fluid.

As used herein, the term “MoVOₓ catalyst” refers to a mixed metal oxide having the empirical formula Mo_{6.5-7.0}V₃O_{d}, where d is a number to at least satisfy the valence of any present metal elements; a mixed metal oxide having the empirical formula Mo_{6.25-7.25}V₃O_{d}, where d is a number to at least satisfy the valence of any present metal elements, or combinations thereof.

As used herein, the term, “selective oxidation” or “SO” refers to an oxidation process that does not proceed to complete thermodynamic oxidation, for example, stopping at products more complex than carbon dioxide and water. As used herein, “oxidative dehydrogenation” or “ODH” is a subset of selective oxidation and refers to processes that couple the endothermic dehydrogenation of an alkane with the strongly exothermic oxidation of hydrogen as is further described herein.

In some embodiments disclosed herein, the degree to which carbon monoxide is produced during an SO process can be mitigated by converting it to carbon dioxide, which can then act as an oxidizing agent. The process can be manipulated so as to control the output of carbon dioxide from the process to a desired level. Using the methods described herein a user may choose to operate in carbon dioxide neutral conditions such that surplus carbon dioxide need not be flared or released into the atmosphere.

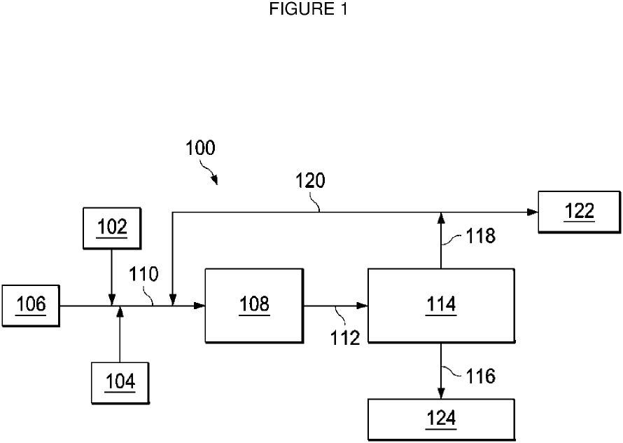

FIG. 1 is a block diagram of a selective oxidation system 100 for the production of acetic acid from the selective oxidation of light hydrocarbons in the presence of water, in accordance with examples. In this example, the feedstocks include carbon dioxide (CO₂) 102, light hydrocarbons 104, and water (H₂O) 106. The feedstocks 102, 104 and 106 are added to a selective oxidation (SO) reactor 108 as a feed stream 110 for conversion of at least a portion of the feedstocks to acetic acid. In various embodiments, the feedstocks 102, 104, and 106 are as individual feed streams, or in various combined feed streams with each other and recycled feedstock streams

In some embodiments, the CO₂ 102 is provided by a petrochemical source, such as an ODH reactor, a cracker, or a water-gas shift reactor. In some examples, the CO₂ 102 is provided to the SO reactor 108 as an exhaust stream, or flue gas, from a combustion process. This could be the flue gas from a gas fired turbine, a boiler used for steam generation, or any number of other combustion processes. As water is formed during the combustion process, the flue gas will also provide at least a portion of the water used in the SO reactor 108. Accordingly, the amount of steam used as the H₂O 106 may be adjusted to achieve the desired selectivity. In some examples, CO₂ 102 is provided to the SO reactor 108 from a carbon dioxide pipeline.

In some embodiments, the H₂O 106 is provided as low-pressure steam from a petrochemical source. This may include steam generated by an ODH reactor or steam from a boiler in a refinery. The H₂O 106 is generally added as superheated, or dry, steam to prevent catalyst pulverization caused by water flashing inside the catalyst bed to avoid damaging the bed. The amount of steam added to the SO reactor 108 may be adjusted to control the selectivity. Generally, higher amounts of water added increase the amount of acetic acid formed in the reaction.

In some examples, the light hydrocarbons 104 may include ethane, ethylene, or a mixture thereof. The light hydrocarbons 104 may be provided by any number of refinery or petrochemical processes. In various embodiments, the light hydrocarbons 104 are provided by a cracking unit, or an ODH reactor, or both. In various embodiments, the light hydrocarbons 104 are provided from a natural gas plant.

The reaction scheme shown below shows reactions that may be taking place in the SO reactor 108. It should be kept in mind that these reactions are merely examples as the reaction chemistry is complicated. Accordingly, the anticipated bulk reaction (6) cannot be balanced based on simply summing up the previous reactions (1) - (5), indicating the complexity of the chemistry. For simplicity, reaction (6) is estimated by summing up reactions (1), (2) and (3).

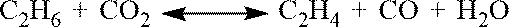

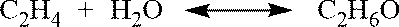

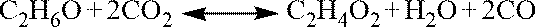

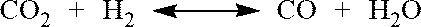

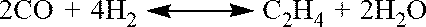

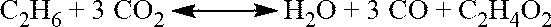

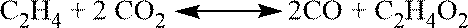

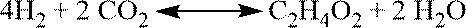

Reaction (7) is generally present when ethylene is used as at least a portion of the light hydrocarbon 104, although a portion of the ethylene formed in reaction (1) may be consumed in reaction (7). Reaction (7) is estimated by summing up reactions (2) and (3). Based on the experiments described in the examples, it can be inferred that occurrence of the reaction (6) is contingent to presence of steam in the feed mixture of the reactor. In the absence of steam, the reaction does not proceed.

As described with respect to the examples below, in addition to acetic acid, trace amounts of CO and ethylene may be present in the product stream when ethane or an ethane/ethylene mixture is used as the feed. The amount of these trace components is less than about 0.1 mol%. In the reported examples, the amount of detected CO was low, for example, either zero or at least one order of magnitude lower than the amount of acetic acid. The CO may be converted back to CO₂, for example, by the reverse of reactions (1) or (4) at the tested operating conditions and feed compositions, although this implies no CO₂ consumption. Further, some of the CO may get consumed in reaction (5), pushing the equilibrium of reaction (1) and (4) toward higher CO₂ consumption. Accordingly, this may result in reaction (8), which is the sum of reactions (2), (3) and (5). Reaction (8) also provides an explanation for the conversion of a CO₂/H₂ into acetic acid.

In the standalone case, the carbon dioxide may be provided from any number of petrochemicals sources such an oxidative dehydrogenation (ODH) process or a cracking process, among others. The ethane feed can come from any number of petrochemical sources such as a natural gas processing plant, a separation system, downstream of a cracker, a C₂ splitter, and the like.

The reaction is facilitated by an SO catalyst. In some examples, the SO catalyst has the formula shown in equation 1 (Eqn. 1).

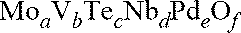

In this formula a, b, c, d, e, and f are relative atomic amounts of the elements Mo, V, Te, Nb, Pd, and O, respectively. When a is 1, b is between 0.01 and 1.0, c is between 0 and 1.0, d is between 0 and 1.0, e is between 0 and 0.10, and f is a number to at least satisfy the valence state of any metal elements present in the SO catalyst. Another example of a catalyst that may be used in processes is shown in the formula of equation 2 (Eqn. 2).

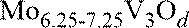

In equation 2, d is a number to at least satisfy the valence of the any present metallic elements in the catalyst. In other examples, the SO catalyst includes vanadium in addition to any other components, such as mixed metal oxides, that are present.

As used herein, the phrase “to at least satisfy the valence” indicates that additional oxygen may be present. In some embodiments, oxygen is absorbed into the catalyst as lattice oxygen. The lattice oxygen may participate in the catalytic reactions through the transfer of oxygen to the hydrocarbon substrate. This temporarily generates a vacancy, or defect, in the catalyst that is replenished by other oxygen atoms or molecules absorbed in the lattice or external to the catalyst. Accordingly, the catalyst can rely on the ability of the metal oxide to form phases that are not stoichiometric to promote reactions.

In some embodiments, oxygen may be present on the surface of the catalyst in the form of hydroxyl groups. In these embodiments, the amount of oxygen for at least a portion of the metals present is double the amount needed to satisfy the valences of the metals as a hydrogen atom is also present.

Accordingly, there is generally more oxygen in the catalyst than is required to satisfy the valences of the metals. The measurement of the oxygen may be performed by any number of different techniques, such as the use of inductively coupled plasma-mass spectrometry (ICP-MS) to infer oxygen content by measuring the content of other elements and subtracting the total from 100%. A direct technique for measuring oxygen content is inert gas fusion, in which the sample is heated by electrodes in a graphite crucible under an inert gas flow. The oxygen present beyond that needed to satisfy the valence states is a combination of lattice oxygen and hydroxyl groups, as well as any other oxygen containing groups, such as carbonate groups and the like.

In some examples, the catalyst may be supported on, or agglomerated with, a binder. Some examples of binders include acidic, basic, or neutral binder slurries of TiO₂, ZrO₂, Al₂O₃, AlO(OH) and mixtures thereof. Another useful binder includes Nb₂O₅. The agglomerated catalyst may be extruded in a suitable shape, such as rings, spheres, saddles, cylinders, and the like of a size typically used in fixed bed reactors. When the catalyst is extruded, various extrusion aids known in the art can be used. In some cases, the resulting support may have a cumulative surface area, as measured by BET, of less than about 35 m²/g, less than about 20 m²/g, or less than about 3 m²/g, and a cumulative pore volume from about 0.05 to about 0.50 cm³/g.

The reactor effluent 112 includes acetic acid, and may include C₁-C₆ oxygenates, CO, unreacted CO₂, unreacted C₂H₄, unreacted CO₂, unreacted C₂H₆, and unreacted H₂O. The reactor effluent 112 sent to a scrubber 114, for the removal of acetic acid, the C₁-C₆ oxygenates, and water. The scrubber 114 may include any number of configurations, such as a heat exchanger followed by a flash vessel or quench tower, which condenses the liquids 116 formed in the SO reactor 108 and separates them from gases 118.

The gases 118 separated from the liquids 116 may include CO₂, light hydrocarbon, CO, C₂H₄ and C₂H₆. A portion of the gases 118 may be returned to the SO reactor 108 as a recycle feed 120 that is combined with one or more of the feedstocks 102, 104 and 106. As indicated by the examples, the ethane conversion rate for a single pass is low, for example, less than about 2 C-atom %. Thus, increasing the fraction of the recycle feed 120 that is returned to the SO reactor 108 may increase the total conversion of the CO₂ 102 and light hydrocarbon 104 to acetic acid. In examples, the conversion rate of CO₂ 102 and light hydrocarbon 104 in a single pass through the SO reactor 108 is about 15 C-atom %, 10 C-atom %, 5 C-atom %, 2 C-atom %, or lower, depending on the flow rate through the SO reactor 108. These values apply for either the conversion of light hydrocarbon or CO₂ to acetic acid.

In some examples, the gases 118 may be provided to other processes as a mixed light hydrocarbon/CO₂ product 122, for example, to be used as a fuel in a combustion process. The liquids 116 include a mixture 124 of acetic acid and H₂O, which may be directly provided as a product or further purified to increase the concentration of the acetic acid for sale. In some examples, at least a portion of the water removed during the purification of the acetic acid may be returned to the process feed, for example, being combined with the H₂O 106.

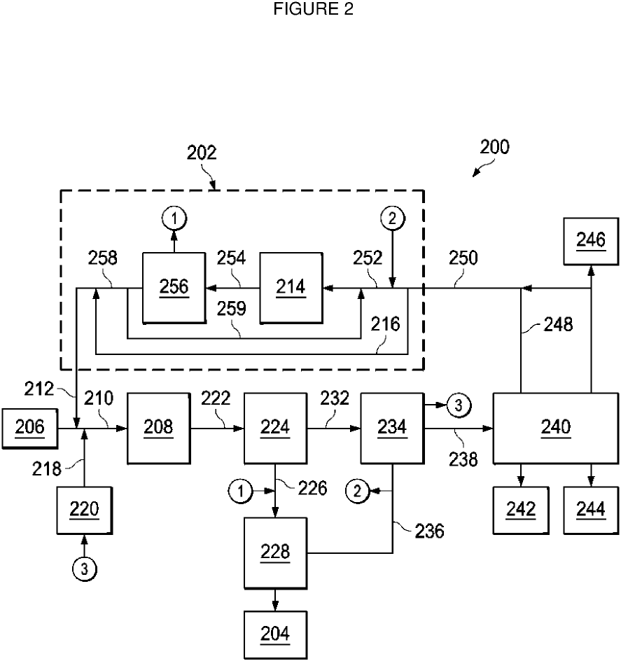

FIG. 2 is a block diagram of a chemical complex 200 that integrates a selective oxidation (SO) system 202 into the chemical complex 200 for the production of acetic acid 204 from the selective oxidation of ethane and ethylene in the presence of water, in accordance with examples. In this example, a feed stream 206 may include CO₂, O₂, and ethane, or other light hydrocarbons. The CO₂ and light hydrocarbon may be as described with respect to the CO₂ 102 and light hydrocarbon 104 of FIG. 1.

The feed stream 206 is provided to an ODH reactor 208, for example, as reactor feed 210. Reactor feed 210 may include an integrated stream 212 from the SO system 202. If an SO reactor 214 in the SO system 202 is online, the integrated stream 212 will include CO₂, C₂H₆, CO and C₂H₄. If the SO reactor 214. In the SO system 202 is not online, a bypass line 216 may be used to provide a recycle feed as the integrated stream 212. As discussed further herein, the recycle feed will include CO₂ and C₂H₆, regardless of whether the SO reactor 214 is online. Further, the reactor feed 210 may include an acetic acid/water stream 218, provided from a vaporizer 220.

The ODH reactor 208 generates an ODH effluent stream 222 that includes ethylene, ethane, O₂, CO, CO₂, H₂O and acetic acid. The ODH effluent stream 222 is provided to an O₂ removal system and condenser 224. After removing the O₂, the condenser condenses the liquids 226 from the ODH effluent stream 222, including, acetic acid, C₁-C₆ oxygenates, and H₂O. The liquids 226 are provided to an acetic acid separation train 228. In the acetic acid separation train 228, acetic acid 204 is purified for sales as a product.

The gases 232 separated in the O₂ removal system and condenser 224, are sent to a scrubber 234, along with a steam stream 236 from the acetic acid separation train 228. The scrubber 234 removes remaining traces of the acetic acid and water from the gases 232 and steam stream 236. The acetic acid and water removed by the scrubber 234 may be used as feed 3 provided to the vaporizer 220, and used for a portion of the reactor feed 210. The small amount of acetic acid in feed 3 may be sufficient to shift the kinetics of the reaction in the ODH reactor 208, increasing the amount of ethylene produced in the ODH reactor 208. The gases 238 from the scrubber 234, are provided to a separation train 240.

The separation train 240 may include cryogenic distillation systems, C₂ splitters, and the like, to isolate a number of components from the gases 238 from the scrubber 234. These components may be recycled to the process, at least in part, or provided as separate product streams. For example, a fuel product 242, including CO, CH₄ and diluent (other than CO₂ and H₂O) may be sold or combusted to provide utilities, such as steam. A C₂H₄ product stream 244 may be isolated from a C₂ splitter, and sold or used to form polyolefins. A CO₂ product stream 246 may be isolated and sold or emitted.

A portion of the CO₂ product stream 246 may be combined with an ethane stream 248 from the C₂ splitter of the separation train 240 to form a mixed stream 250 that is provided to the SO system 202. A portion of the steam stream 236, identified as feed 2, is combined with the mixed stream 250 to form a feed stream 252 for the SO reactor 214. If needed, additional low-pressure steam may be added to feed 2 to increase the water content of the feed stream 252.

The effluent 254 from the SO reactor 214 includes acetic acid, C₁-C₆ oxygenates, CO, C₂H₄, CO₂, C₂H₆ and H₂O, among other components. The effluent 254 is sent to a condenser 256. The condenser 256 generates a liquid stream that includes the acetic acid, the C₁-C₆ oxygenates, and the H₂O, which is provided as feed 1. Feed 1 is combined with the liquids 226 from the ODH effluent stream 222, prior to being sent to the acetic acid separation train 228. The gases 258 from the condenser 256 may be used as the integrated stream 212 when the SO reactor 214 is online.

Further, the gases 258 from the condenser 256 may be recycled to the SO reactor 214 through an SO recycle line 259. The SO recycle line 259 can be used to recycle portion of the unreacted ethane-CO₂ into the inlet of reactor 214 to increase conversion towards acetic acid. As described herein, when the SO reactor 214 is not online, a bypass line 216 is used to direct the mixed stream 250 around the SO reactor 214 and the condenser 256, forming the integrated stream 212.

In this process configuration, the mixed stream 250 includes recycled CO₂ and unreacted ethane which is used in the SO reactor 214 to generate acetic acid. The acetic acid may then be provided as a portion of the reactor feed 210 to the ODH reactor 208. Thus, a portion of the ethane stream 248 is converted to acetic acid and recycled back to the ODH reactor 208. The presence of the acetic acid in the feed effluent of the ODH reactor 208, may suppress or reduce acetic acid formation in the ODH reactor 208, increasing the ethylene formation. The process integration also provides heat integration. As reaction (6) is endothermic, the heat generated from the ODH reactor 208 provides heat for the endothermic reaction that generates acetic acid from Ethane/CO₂/H₂O in the so reactor 214, increasing the energy efficiency of the process.

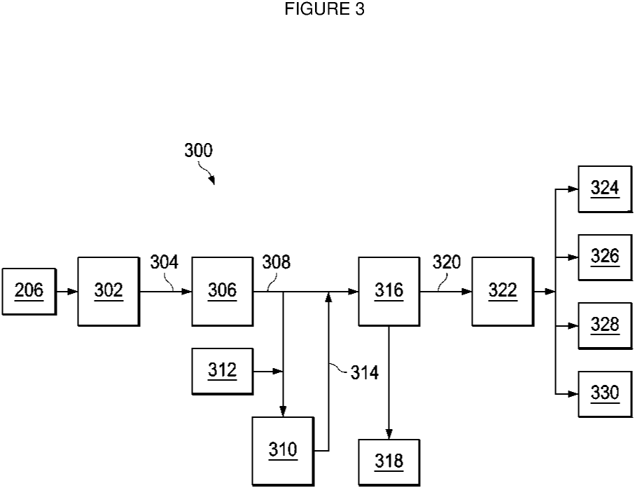

FIG. 3 is a block diagram of a selective oxidation system 300 for the production of acetic acid from the selective oxidation of a mixture of ethane and ethylene in the presence of water, in accordance with examples. Like numbered items are as described with respect to FIGS. 1 and 2. As described with respect to FIG. 2, the feed stream includes C₂H₆, O₂ and a diluent, such as CO₂. It may be noted that the diluent may be inert in terms of flammability but is a reactant when contacted with the catalyst of the ODH process.

The feed stream 206 is provided to an ODH reactor 302. The ODH reactor 302 generates an effluent stream 304 that includes acetic acid, C₂H₆, C₂H₄, CO, CO₂, O₂ and H₂O. The effluent stream 304 is provided to an oxygen removal system 306. In the oxygen removal system 306, trace oxygen is removed to a level below about 50 ppmv, below about 25 ppmv, below about 10 ppmv, below about 5 ppmv, or below about 2 ppmv. In some embodiments, the oxygen removal system 306 utilizes an oxygen separation membrane at a high temperature, as described herein. In other embodiments, the oxygen removal system 306 uses a catalytic conversion system to convert the oxygen and CO to CO₂.

The deoxygenated effluent 308 includes acetic acid, C₂H₆, C₂H₄, CO, CO₂ and H₂O. At least a portion of the deoxygenated effluent 308 is sent to an SO reactor 310, as a portion of a feed stream to the SO reactor 310. An additional feed stream 312, including CO₂ and H₂ is combined with the deoxygenated effluent 308 that is sent to the SO reactor 310. The CO₂ and H₂O of the additional feed stream 312 increases the hydrocarbon conversion and CO₂ conversion to the value-added acetic acid product. The H₂ can come from an external source, such as an ethane cracker plant, among others. The CO₂ can come from an ODH CO₂ recycle stream or an external source, such as an ethane cracker flue gas, a utility flue gas, or a pipeline.

The use of the additional H₂ in the feed stream to the SO reactor 310 to increase hydrocarbon conversion and CO₂ conversion is suggested by the comparison of the results from example 1 to the results from example 4. The addition of H₂ was found to increase the conversion of CO₂ conversion. Further, additional CO₂ may be added to the feed stream of the SO reactor 310 to take advantage of the additional CO₂ conversion caused by presence of H₂ in the feed. Generally, addition of the H₂ to the feed stream of the SO reactor 310 increases the hydrocarbon conversion and CO₂ conversion in this reactor, although the addition of CO₂ to this feed effluent is optional.

The SO reactor 310 forms additional acetic acid, forming an effluent stream 314 that includes acetic acid, C₁-C₆ oxygenates, CO, CO₂, H₂O, C₂H₄ and C₂H₆. The effluent stream 314 is combined with the remaining portion of the deoxygenated effluent 308 and provided to a scrubber 316. The scrubber 316 uses a condenser to condense the higher boiling point components of the effluents 308 and 314, forming a liquid product 318 that includes acetic acid, H₂O, and C₁-C₆ oxygenates. The scrubber 316 also forms a gas stream 320 that includes lower boiling point components. The gas stream 320 includes C₂H₆, C₂H₄, CO and CO₂.

The gas stream 320 is provided to a gas product separation train 322. The gas product separation train 322 may be used to separate four gaseous streams. An effluent stream 324, including C₂H₆, may be recycled to the feed stream 206. A C₂H₄ stream 326 may be sent to a polyolefin facility to form plastics. A CO stream 328 may be provided to another plant or combined with a fuel stream for combustion, for example, in a plant utility system or a cracking system, among others. Finally, a CO₂ stream 330 may be recycled to the feed stream 206, released to the atmosphere, or provided for further purification as a product stream.

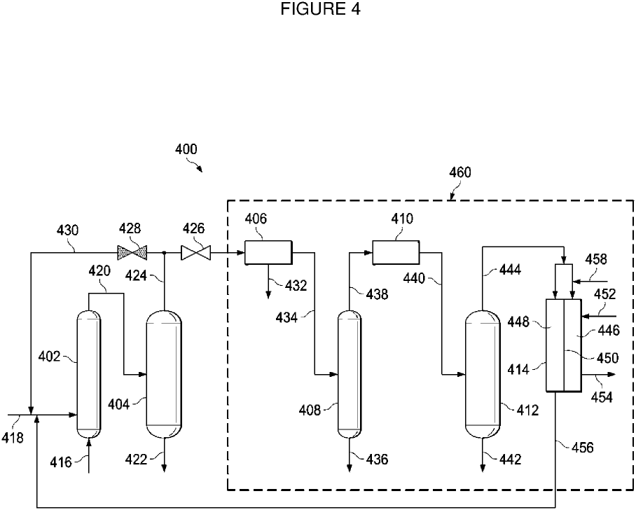

FIG. 4 is a simplified process flow diagram of a chemical complex 400 used for the production of ethylene in an oxidative dehydrogenation (ODH) reaction, in accordance with examples. In this example, the chemical complex 400 includes an SO reactor 402, a scrubber 404, a second reactor 406, an amine wash system 408, a dryer 410, a distillation tower 412, and an oxygen separation module 414. It can be understood that each of these units may include one or more vessels and supporting equipment, such as valves, pumps, sensors, and associated control equipment, such as distributed control systems, and the like.

The arrangement of these units is not limited to the arrangement shown. In some examples, the second reactor 406 is not placed directly downstream of the scrubber 404 but is placed further downstream. Depending on the installation environment, not all of the units shown may be present. Further, additional units may be present, for example, a compressor may be placed upstream of the second reactor 406 to increase the pressure of the feed. In some examples, multiple SO reactors are used in a parallel configuration, as described with respect to FIG. 4.

The SO reactor 402 may be a fixed bed reactor or a fluidized bed reactor. The SO reactor 402 includes an SO catalyst capable of catalyzing the oxidative dehydrogenation of alkanes introduced through an alkane line 416. The reaction takes place in the presence of oxygen, which may be introduced through a feed line 418. The feed line 418 may be a single feed line with a combination of the feed stocks or may be divided into any combinations of a light hydrocarbon feed line, a carbon dioxide feed line, and a steam feed line. The SO reaction may also occur in the presence of an inert diluent, such as carbon dioxide, nitrogen, or steam. The inert diluent may be added to the mixture to lower the flammability of the mixture during the SO reaction, or to add additional reactants for the SO reaction used to produce acetic acid, as described herein. It may be noted that the inert diluent is defined with respect to flammability of the reactants. Some of the inert diluents, such as CO₂, may participate in the catalytic reaction in the SO reactor 402.

In some examples, all of the reactants are added through the feed line 418. In these examples, upstream equipment may be used to blend the reactants below flammability limits prior to introduction to the SO reactor 402. In some examples, a flooded gas mixer is used to allow mixing of the gases while they are surrounded by a non-flammable liquid, such as water.

The ODH reaction that occurs within the SO reactor 402 may also produce a variety of other products in addition to ethylene and other target olefins. The other products may include carbon dioxide, carbon monoxide, oxygenates, such as acetic acid, and water. These reaction products from the SO reactor 402 are carried by the SO effluent line 420 to the scrubber 404 along with unreacted alkane, the corresponding alkene, residual oxygen, carbon monoxide, and inert diluent. The scrubber 404 quenches the products in the effluent from the SO reactor for the removal of oxygenates and water through the scrubber bottom outlet 422.

The gases that are separated from the reaction products exit the scrubber 404 through the scrubber overhead line 424. The gases may include unconverted alkanes, corresponding alkanes, unreacted oxygen, carbon dioxide, carbon monoxide, and inert diluent. These gases may be directed to the second reactor 406 through an ODH control valve 426 for further processing. As described further herein, an SO recycling valve 428, shown as closed in FIG. 4, is used in other examples to recycle at least a portion of the gases to the fresh feed lines through a recycling line 430.

In this example, the second reactor 406 contains a catalyst with a group 11 metal, for example, with a promoter and support, to react oxygen in the gases with carbon monoxide to form carbon dioxide. The second reactor 406 may be a fixed bed reactor or a fluidized bed reactor. Further, the catalyst can react acetylene with oxygen to reduce or eliminate it. The carbon dioxide from the second reactor 406 can be recycled to the SO reactor 402 through a recycling line 432.

The remaining gases, including a portion of the carbon dioxide, unconverted lower alkanes, the corresponding alkenes, and any other remaining materials are conveyed to the amine wash system 408 through a products line 434. The amine wash system 408 may include an absorber tower in which the gases are contacted with a lean amine, such as diethanolamine, monoethanolamine, or methyldiethanolamine, among others. The majority of the carbon dioxide in the gases is then captured by reaction with the lean amine. A caustic tower may be located downstream for further polishing and removal of trace amounts of CO₂ that may have past amine tower.

After the amine captures the carbon dioxide, it is termed a rich amine. The rich amine is sent to a regenerator in which the carbon dioxide is removed from the rich amine, providing the lean amine that is returned to the absorber tower of the amine wash system 408, and a carbon dioxide stream. The carbon dioxide stream exits the amine wash system 408 through a carbon dioxide outlet 436. In some examples, the carbon dioxide is recycled back to the SO reactor 402 or sold as a product stream. Components of the gases that are not absorbed in the amine wash system 408 exit through an absorber overhead line 438 which conducts the components to the dryer 410.

In various examples, the dryer 410 is a multistage chiller and cryogenic dryer that removes water through a condensation process in a first stage, and then successively removes remaining amounts of water in following stages. In other examples, the dryer 410 is an adsorption, which absorbs water by flowing the gases through zeolites or other materials. The dried gas is conducted from the dryer 410 through a dry gas line 440 to the distillation tower 412.

The distillation tower 412 may include a single vessel or multiple vessels that perform cryogenic separation. In the distillation tower 412 C2/C2+ hydrocarbons are separated and removed through a distillation bottom outlet 442. The remaining gases include mainly methane, inert diluent, such as nitrogen, and any remaining carbon monoxide. These gases leave the distillation tower through a distillation top outlet 444 and are directed to the oxygen separation module 414.

In this example, the oxygen separation module 414 includes a sealed vessel having a retentate side 446 and a permeate side 448, separated by an oxygen transport membrane 450. As shown, the gases from the distillation top outlet 444 may be directed either to the retentate side 446 or the permeate side 448. In some examples, flow controllers may be included to allow for flow into both sides at varying levels. The oxygen separation module 414 also includes an air input 452 to introduce an oxygen -containing gas into the retentate side 446. Combustion of products in the gases from the distillation top outlet 444 may then heat the oxygen transport membrane 450 to greater than about 850° C., allowing oxygen to pass from the retentate side 446 to the permeate side 448. The oxygen transport membrane 450 blocks other components, besides oxygen, which exit the retentate side 446 of the oxygen separation module 414 through an exhaust 454.

Accordingly, the oxygen separation module 414 provides an oxygen enriched gas from the permeate side 448 that exits the oxygen separation module 414 through an oxygen return line 456. The oxygen return line 456 is then coupled to the feed line 418, or upstream blending equipment, for return to the SO reactor 402. When the gases from the distillation top outlet 444 are directed into the retentate side 446, the concentration of the oxygen in the oxygen return line 456 can approach 99%, or higher. When the gases from the distillation top outlet 444 are directed into the permeate side 448, the concentration of the oxygen in the oxygen return line 456 may be about 80% to about 90%, with the remaining gases being carbon dioxide, water, and inert diluent. If the combustion of products in the gases from the distillation top outlet 444 are not sufficient to raise the oxygen transport membrane 450 to this temperature, fuel may be added through a fuel line 458.

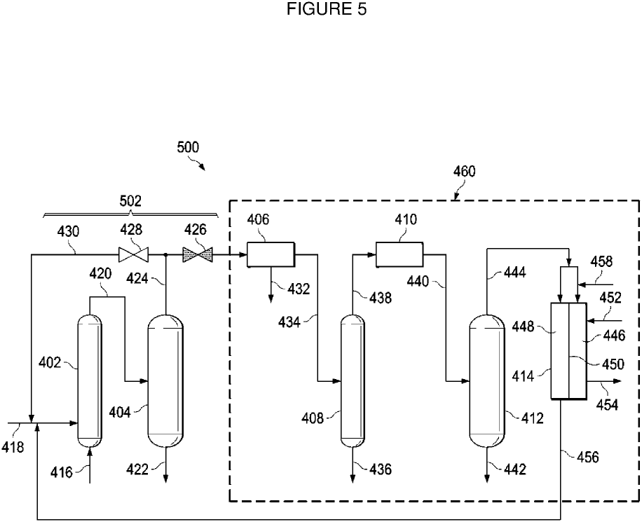

The ODH downstream separation processing taking place in vessels 406, 408, 410, 412 and 414, and their associated equipment, may be grouped into an ODH system 460, as shown in FIG. 4. As described herein, the ODH control valve 426 may direct at least a portion of the gases from the scrubber overhead line 424 to the ODH system 460 for further processing, for example, to sell C2/C2+ from the distillation bottom outlet 442 as a product. The SO recycling valve 428, shown as closed in FIG. 4, is used in other examples to recycle at least a portion of the gases to the fresh feed lines through a recycling line 430, as described further with respect to FIG. 5.

FIG. 5 is a simplified process flow diagram of an SO reactor system 500 for the production of acetic acid, in accordance with examples. Like numbered items are as described with respect to FIG. 4. In this example, the ODH control valve 426 is shown as closed, and the SO recycling valve 428 is shown as open, thus recycling all of the gases from the scrubber overhead line 424 back to the feed line 418. Accordingly, in this example, the reactor 402 and scrubber 404 are functioning as an acetic acid reactor system 502. In the acetic acid reactor system 502 the product is acetic acid produced from the scrubber 404 through the scrubber bottom outlet 422, which is the acetic acid product line.

In some examples, the ODH control valve 426 and the SO recycling valve 428 are proportionally controlled to allow a portion of the gases from the scrubber overhead line 424 to be returned to the SO reactor 402, and another portion of the gases from the scrubber overhead line 424 to be sent on for further processing in the ODH system 460. The acetic acid produced from the scrubber 404 may be processed for sale, as described herein, or may be used to generate further products, such as ethanol or ethylene. In one example, the acetic acid is converted to ethylene by processing it in a second SO reactor system feeding an ODH system. The addition of the acetic acid to the second SO reactor system provides a negative selectivity towards further acetic acid production and an increase in selectivity of ethylene. As used herein, a negative selectivity means that acetic acid is consumed in the process, resulting in increased ethylene production per unit of consumed ethane in the same process.

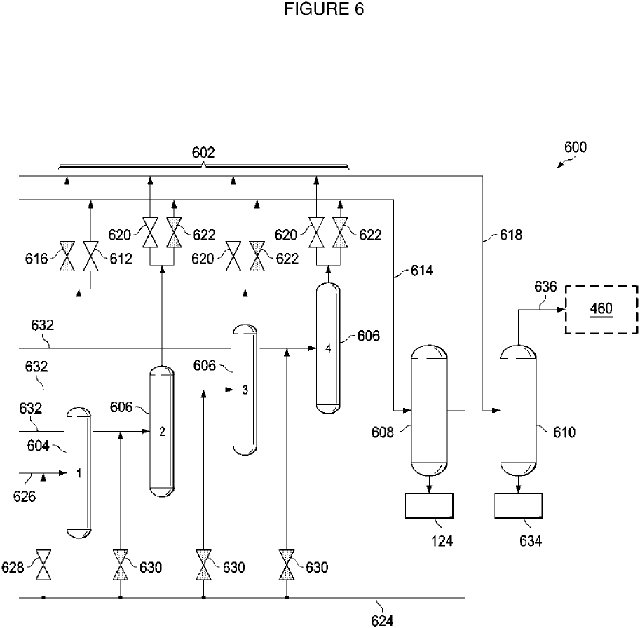

In various examples, a number of SO reactors are used in a parallel configuration, with at least a portion of the ODH system 460 shared by the SO reactors. In this example, a portion of the SO reactors can be used to produce acetic acid, while other SO reactors may provide feed to the ODH system 460. This configuration is described further with respect to FIG. 6.

FIG. 6 is a simplified process flow diagram 600 for using a group 602 of parallel SO reactors wherein one SO reactor 604 is used in a swing capacity to produce acetic acid, while other reactors 606 are producing ethylene, in accordance with examples. Like numbered items are as described with respect to FIGS. 1 and 4.

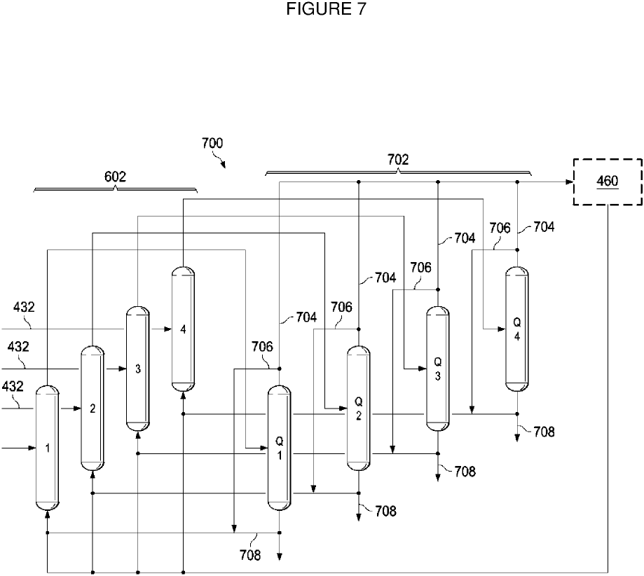

In this example shown in FIG. 6, the group 602 has four parallel SO reactors that can be fluidically coupled to either one of two downstream scrubbers, an acetic acid scrubber 608, or an ODH scrubber 610. This illustration is a simplification to allow details to be seen. Generally, each SO reactor will have its own scrubber, as shown in the last detailed illustration of FIG. 7.

In this example, reactor 1 604 is coupled to the acetic acid scrubber 608 through a valve 612 that is open to an acetic acid header 614. A second valve 616 between reactor 1 604 and an ODH header 618 is closed. The other reactors 606 in this example, reactor 2, reactor 3 and reactor 4, are coupled to the ODH header 618 through open valves 620, and isolated from the acetic acid header 614 through closed valves 622. Further, a scrubber gas recycle header 624 is coupled to the feed line 626 for reactor 1 604 through a valve 628 that is open to the scrubber gas recycle header 624, while the scrubber gas recycle header 624 is isolated from the remaining reactors 606 by valves 630 that are closed.

Configurations are not limited to the arrangements of the vessels shown. In some examples, a separate ODH scrubber 610 is provided for each of the reactors, and an acetic acid scrubber 608 is provided to allow one of the reactors to be switched from ethylene production to acetic acid production. In other configurations, multiple vessels are used in place of each of the scrubbers 608 and 610. In these examples, a condenser vessel is used to separate liquid and gas from the reactor effluent. Gas from the condenser is combined with an overhead gas from the liquid and processed in an acetic acid scrubber to remove further traces.

In the example shown, the feed line 626 for reactor 1 may carry a feed that includes a light hydrocarbon, carbon dioxide, and steam, providing a high selectivity for the formation of acetic acid in reactor 1. As described herein, the light hydrocarbon may be ethane or a mixture of ethane and ethylene. Thus, in this configuration, reactor 1 604 functions as an SO reactor producing acetic acid, as described with respect to the SO reactor 108 of FIG. 1. The feed lines 632 to the remaining reactors 606 carry a feed used for an ODH process, such as ethane, oxygen, and carbon dioxide.

The acetic acid isolated from the mixture 124 of acetic acid and H₂O removed in the acetic acid scrubber 608 may be sold as a separate product. In some examples, the acetic acid may be combined with the feed to the remaining reactors 606, and added through feed lines 632. As described herein, this may increase the production of ethylene by increasing the selectivity for ethylene over acetic acid in an ODH reaction.

The products from the remaining reactors 606 are carried by the ODH header 618 to the separate ODH scrubber 610. Oxygenates and water 634 are isolated from the separate ODH scrubber 610 as described with respect to the scrubber 404 of FIG. 4. A scrubber overhead line 636 from the separate ODH scrubber 610 feeds the gases to the ODH system 460 for further processing, as described with respect to FIG. 4.

As described herein, the valves 612 and 616 coupling reactor 1 604 to the acetic acid header 614 and the ODH header 618 do not have to be fully open or fully closed. Instead, these valves 612 and 616, and the valve 628, that couples the feed line 626 for reactor 1 604 to the scrubber gas recycle header 624 may be control valves that are partially open or partially closed, as determined by a control system. This may allow a different mixture of products to be formed in the system.

FIG. 7 is a simplified process flow diagram of a reactor system 700 for using a group of parallel SO reactors wherein one SO reactor is used in a swing capacity to produce acetic acid, while other SO reactors are producing ethylene, in accordance with examples. Like numbered items are as described with respect to FIG. 4. In this example, each reactor (1-4) in the reactor group 602 has a dedicated scrubber (Q1-Q4) in the scrubber group 702. To simplify the drawing, the individual control valves for each reactor and scrubber are not shown, however, the gas outlet for each scrubber (Q1-Q4) can be fluidically coupled to the downstream ODH system 460 through an ODH line 704. Similarly, the gas outlet for each scrubber (Q1-Q4) can be fluidically coupled through a gas recycle line 706 to recycle feed to the corresponding reactor (1-4). A liquid recycle line 708 may be used to recycle a portion of the liquid separated from a scrubber (Q1-Q4) back to a corresponding reactor (1-4).

In some embodiments, the gas recycling line 706 or the liquid recycling line 708 from a reactor (1-4) is configured to allow material to be recycled from the corresponding scrubber (Q1-Q4) to another one, or all, of the other reactors. For example, reactor 1 and scrubber Q1 may be configured as an SO reactor to form acetic acid, and a portion of the liquid from scrubber Q1 may be recycled to reactors 2-4 to enhance selectivity for ethylene production. This arrangement would allow the configuration of reactors shown in FIG. 2.

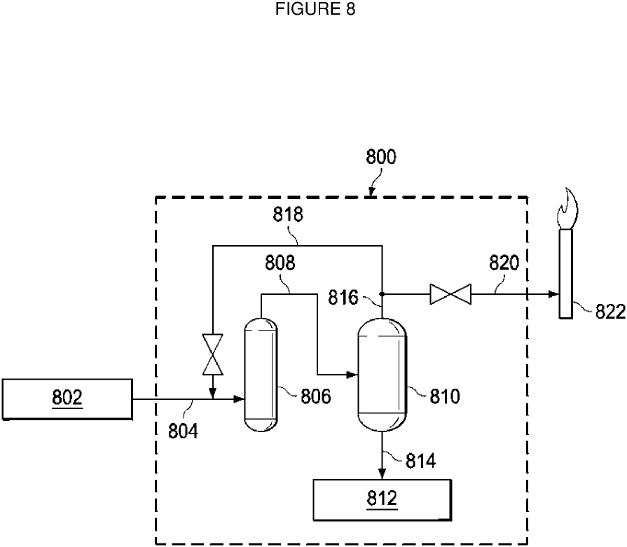

FIG. 8 is a simplified process flow diagram of a process skid 800 for the production of acetic acid from a light hydrocarbon and carbon dioxide, in accordance with examples. The process skid 800 allows an acetic acid production reactor to be placed in convenient locations, for example, at natural gas plants, refineries, and the like. The feeds 802 to the process skid 800 may include a light hydrocarbon, carbon dioxide, and steam. The light hydrocarbon may be ethane, ethylene, or a mixture thereof. As described herein, the ethane may be obtained from a natural gas plant, a cracker, or another source and the carbon dioxide and steam may be at least partially sourced from a flue gas provided from a combustion process.

The feeds 802 may be combined in the feed line 804, which is provided to the SO reactor 806. As described with respect to the SO reactor 108 of FIG. 1, the SO reactor 806 produces acetic acid at a single pass yield of about 10 C-atom % with a reactor effluent that includes acetic acid, unreacted light hydrocarbon, unreacted CO₂, and water, among other components. The reactor effluent is carried by an effluent line 808 to a scrubber 810.

As described with respect to the scrubber 114 of FIG. 1, the scrubber 810 quenches the reactor effluent, generating a liquid product 812 that includes acetic acid, water, and any other oxygenated chemicals. The liquid product 812 exits the scrubber 810 through a bottoms line 814, which is the acetic acid product line. Gases isolated from the liquid product 812 exit the scrubber 810 through an overhead line 816. In some examples, the gases are at least partially recycled to the feed line 804 through a recycle line 818 to increase the yield. A portion of the gases may be sold or discarded through a gas outlet line 820, for example, being sent to a flare 822, or used in a combustion turbine for power generation or used as a feed effluent diluent for an ODH reactor.

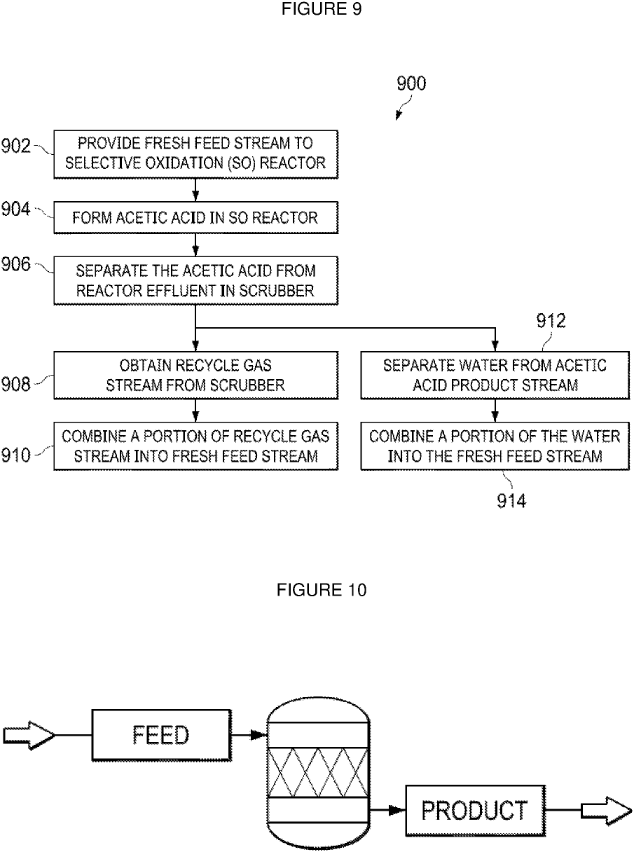

FIG. 9 is a block flow diagram of a method 900 for the production of acetic acid in a selective oxidation reactor, in accordance with examples. The method 900 begins at block 902, when a fresh feed stream is provided to a selective oxidation (SO) reactor. As described herein, the fresh feed stream may include a light hydrocarbon, such as ethane, carbon dioxide and water. The reaction may produce acetic acid, ethylene, carbon monoxide and carbon dioxide. This may be convenient in chemical complexes that already have an ethane feed to the reactors.

At block 904, acetic acid is formed in the SO reactor, for example, using the catalysts and processes described herein. At block 906, acetic acid is separated from the reactor effluent in a scrubber, as a liquid stream removed from the bottom of the scrubber. At block 908, water is separated from the acetic acid product, for example, to increase the concentration of the acetic acid for sale. At block 910, a portion of the water may be combined into the fresh feed stream provided to the SO reactor.

At block 912, a recycle gas stream is isolated from the scrubber. The recycle gas stream may include unreacted methane and carbon dioxide. At block 914, a portion of the recycle gas stream is combined into the fresh feed stream to the reactor. As described herein, this increases the total yield of the process.

### EXAMPLES

The production of acetic acid in a SO reactor was tested using a fixed bed reactor unit (FBRU). The FBRU apparatus comprised two vertically oriented fixed bed tubular reactors in series, each reactor a SS316L tube with an outer diameter of 2.54 cm, an internal diameter of 2.1 cm, a length of 86.4 cm, wrapped in an electrical heating jacket, and sealed with ceramic insulating material. Each reactor contained an identical catalyst bed consisting of extruded pellets, in cylinder form having a diameter of 1.7 mm, lengths ranging from 2 to 10 mm, and comprising a mixture of one weight unit of catalyst to 1.22 units of weight of VERSAL™ Alumina 250 powder. Total weight of the catalyst in each reactor was 171 g catalyst having the formula Mo_{1.0}V_{0.37}Te_{0.23}Nb_{0.14}O_{d=4.97}, with relative atomic amounts of each component to a relative amount of Mo of 1, shown in subscript. The composition was based on ICP-MS measurements where “d” was calculated based on the highest oxide state of the metal elements present.

The temperature of each of the reactors was monitored using corresponding 7-point thermocouples present in each reactor, 5 of which were situated within each catalyst bed, the average of which is reported in Table 1. Temperature control, particularly at lower temperatures, was limited and resulted in fluctuations. Both reactors were being controlled for temperature by controlling the pressure and temperature of a circulating closed loop oil bath which feed into jackets surrounding each reactor. Furthermore, the temperature of the catalyst bed in each reactor was monitored and controlled based on maximum values of the thermocouple measurements using the oil bath. The pressure inside the reactors was controlled and adjusted using a back pressure regulator located downstream of a condenser on the reactor effluent line.

In the FBRU unit, two feed gases can be separately fed through the reactors. One feed gas is the mixture of methane, carbon dioxide, and water, and the other feed gas is air. The first feed gas is used for conducting SO reactions, such as ODH, and the air can be used for catalyst regeneration. The flow of gases is controlled by mass flow controllers. The flow of other materials added, such as water or other liquids, may be controlled by a positive displacement pump.

In addition to the feed gases, an oxygenate-water mixture can be co-fed along with the mentioned feed gases into the inlet of the reactors using a pump. The oxygenate-water mixture evaporates at the inlet of the reactors prior to reaching the catalyst bed, providing additional reactants for testing. The oxygenate may include acetic acid, ethanol or methanol.

### Example 1: Conversion Tests of Feed Streams Including CO₂, C₂H₆, and H₂O

The reaction of a feed mixture including CO₂, C₂H₆ and H₂O, using the catalyst described above, was tested in the FBRU. The operating conditions, including reaction temperature and reaction inlet pressure, and feed composition used are shown in Table 1. The flow rate used, either as gas hourly space velocity (GHSV) or weight hourly space velocity (WHSV), is also shown, where GHSV is the ratio of gas flow rate under standard conditions for temperature (25° C.) and pressure (100 kilopascals) to the volume of the active phase of the catalyst, and WHSV is the weight of the total feed flowing per unit weight of the catalyst per hour. The measured catalyst activity and product distributions are reported in Table 2.

In all of the tests, acetic acid was the only product generated in significant amounts. However, in some of the experiments, a trace amount of CO and ethylene (less than 0.1 mol%) was detected in the products stream. The amounts of CO or ethylene detected were at least one order of magnitude smaller than acetic acid and considered to negligible and therefore were not used in the calculations for selectivity and conversion.

Calculations assumed that acetic acid was generated from the reaction of ethane with carbon dioxide based on the following equation:

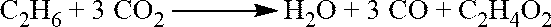

Conversion and selectivity calculations were based on feed reactant molar flow rate/s and equivalent conversion of these reactants during experiments as calculated from acetic acid product molar flow rate and stoichiometric equivalent consumption of the reactants based on reaction above.

Further, an increase in reaction temperature in the range of 250° C. to 320° C., while other reaction and feed parameters were kept unchanged, led into increase in acetic acid yield (hydrocarbon conversion X acetic acid selectivity/100). An increase in reaction inlet pressure in the range of 15 to 52 psig, while other reaction and feed parameters were kept unchanged, led to no change in acetic acid yield (hydrocarbon conversion X acetic acid selectivity/100).

TABLE 1

Reactor Feed Composition Ranges and Operating Condition Ranges

| Test ID | GHSV¹ (h⁻¹) | WHSV² (h⁻¹) | Reaction Temperature (ºC) | Reaction Inlet Pressure (psig) | Feed Composition (Vol %) | | | |
|---|---|---|---|---|---|---|---|---|
| | | | | | H₂O | CO₂ | C₂H₆ | O₂ |
| A | 459 | 0.72 | 250 | 15 | 40 | 25 | 35 | 0 |
| B | 459 | 0.72 | 300 | 15 | 40 | 25 | 35 | 0 |
| C | 459 | 0.72 | 320 | 15 | 40 | 25 | 35 | 0 |
| D | 459 | 0.72 | 300 | 52 | 40 | 25 | 35 | 0 |

TABLE 2

Catalyst Activity and Product Distribution

| Test ID | Reaction Temperature (ºC) | Reaction Inlet Pressure (psig) | Hydrocarbon Conversion (C-atom %) | CO₂ Feed Conversion (C-atom %) | Acetic Acid Selectivity (C-atom %) |
|---|---|---|---|---|---|
| A | 250 | 15 | 0.29 | 1.24 | 100 |
| B | 300 | 15 | 0.71 | 2.99 | 100 |
| C | 320 | 15 | 1.13 | 4.75 | 100 |
| D | 300 | 52 | 0.69 | 2.92 | 100 |

### Example 2: The Effect of Eliminating Steam From the Feed Stream

Test ID E was performed to explore the effect of presence of steam in the reactor feed effluent on catalyst activity and product distribution by using the operating conditions of Test ID C shown in Table 1 above while eliminating steam from the feed (Table 3). The results for Test ID E, shown in Table 4, demonstrate that steam is required to facilitate the conversion of ethane to acetic acid, as no acetic acid was formed in the absence of steam. This is in contrast to Test ID C, which used identical operating conditions but with steam in the feed and resulted in ethane conversion of 1.13% with selectivity to acetic acid of 100%.

TABLE 3

Reactor Feed Composition Ranges and operating condition ranges

| Test ID | GHSV (h⁻¹) | WHSV (h⁻¹) | Reaction Temperature (°C) | Reaction Inlet Pressure (psig) | Feed Composition (Vol %) | | | |
|---|---|---|---|---|---|---|---|---|
| | | | | | H₂O | CO₂ | C₂H₆ | O₂ |
| E | 459 | 0.72 | 320 | 9 | 0 | 42 | 58 | 0 |

TABLE 4

Catalyst Activity and Product Distribution for Feeds Lacking Steam

| Test ID | Reaction Temperature (°C) | Reaction Inlet Pressure (psig) | HC Feed Conversion (C-atom %) | CO₂ Feed Conversion (C-atom %) | Acetic Acid Selectivity (C-atom %) |
|---|---|---|---|---|---|
| E | 320 | 9 | 0 | 0 | N/A |

### Example 3: Conversion Tests of Feed Streams Including CO₂, C₂H₄, H₂, and H₂O

Test ID F was performed to explore the effect of presence of ethylene and/or hydrogen in the feed on catalyst activity and product distribution by using the operating conditions and feed composition of CO₂, C₂H₄, H₂ and H₂O shown in Table 5. The measured catalyst activity and product distributions are reported in Table 6.

The results of Test ID F resulted in conversions of both ethylene (10.0 %) and CO₂ (9%). In the case of ethylene, it is assumed that the selectivity to acetic acid approaches 100%. In the case of CO₂, selectivities acetic acid, methane, and carbon monoxide were calculated to be 85 C-atom %, 1 C-atom % and 13 C-atom %, respectively, assuming that all the C₂H₄ was converted to C₂H₆ and all the CO₂ was converted to CH₃COOH, CH₄ and CO. These results demonstrate that the presence of ethylene and/or hydrogen as potential impurities in the feed stream would not negatively impact the CO₂ conversion to value added products, mainly acetic acid.

TABLE 5

Reactor Feed Composition Ranges and Operating Condition Ranges for Experiments Conducted in this Section

| Test ID | GHSV (h⁻¹) | WHSV (h⁻¹) | Reaction Temperature (°C) | Reaction Inlet Pressure (psig) | Feed Composition (Vol %) | | | |
|---|---|---|---|---|---|---|---|---|
| | | | | | H₂O | CO₂ | C₂H₄ | H₂ |
| F | 456 | 0.45 | 325 | 14 | 19 | 9 | 36 | 36 |

TABLE 6

Catalyst Activity and Product Distribution for Feeds Comprising Ethylene and Hydrogen

| Test ID | Reaction Temp. (°C) | Reaction Inlet Pressure (psig) | Conversion (C-atom %) | | Selectivity (C-atom %) Related to C₂H₄ Conversion | Selectivity (C-atom %) Related to CO₂ Conversion | | |
|---|---|---|---|---|---|---|---|---|
| | | | C₂H₄ | CO₂ | C₂H₆ | CH₃COOH | CH₄ | CO |
| F | 325 | 14 | 10 | 9 | 100 | 85 | 1 | 13 |

Note: CO₂—C₂H₄—H₂O—H₂ test

### Example 4: Simulation Results

In order to identify if the reaction of CO₂ and C₂H₆, via summed Reaction (1), described herein, is endothermic or exothermic, the reaction was simulated. The simulation was performed using Aspen plus software V10, at the operating temperature and pressure of Test ID C. For simplicity, the feed composition was assumed to be only ethane and CO₂ with respective feed mole fraction of 0.25 and 0.75 to satisfy the stoichiometric feed composition requirement to make 100 % conversion of the reactant via Reaction (1). The simulation block flow diagram is shown in FIG. 10. Note that the non-random two liquid (NRTL) property method was used for this simulation. Also, the stoichiometric reactor (RSTOIC) was picked for this simulation. The calculated heat of reaction for Reactor (1) is shown in TABLE 77. The heat required for this reaction can optionally come from ODH reactor. As Reaction (1) is a simplified bulk reaction, it may have some error with respect to true net heat of reaction.

TABLE 7

Calculated Heat of Reaction in the Reactor

| Enthalpy of Reaction (1) (kJ/kmol C₂H₆) | Reactor Heat Duty (kJ/hr) | Feed Ethane Converted in the Reactor (kmol/hr) |
|---|---|---|
| 260973 | 65243.26 | 0.25 |

### Example 5: Conversion Tests of Feed Streams Including CO₂—C₂H₆—C₂H₄—H₂O

The reaction of a feed mixture including CO₂, C₂H₆, C₂H₄, and H₂O, using the catalyst described above, was tested in the FBRU. The operating conditions and feed composition used are shown in Table 8. The measured catalyst activity and product distributions are reported in Table 9.

In all of the tests, acetic acid was the only product generated in significant amounts. However, in some of the experiments, a trace amount of CO, C₁-C₆ liquid oxygenates (less than 0.1 mol%) was detected in the products stream. The amount is at least one order of magnitude smaller than acetic acid and was considered to be negligible and therefore was not used in the calculations for selectivity and conversion.

The results of Test ID G may be compared to Test ID C to determine the effect of ethylene on the process, as the operating conditions for the reactors were relatively similar. Specifically, the GHSV was less than 30% relative difference, the reaction temperature was the same, and the reactor in the pressure was nearly identical. However, in the Test ID G, ethylene is present in the feed stream, while in Test ID C, ethylene is absent from the feed stream.

The catalyst activity and product distribution for the two examples are similar, with less than 1% absolute difference in the reported feed conversion and no difference in acetic acid selectivity. Further, in both test ID C and test ID G the CO₂ conversion and hydrocarbon conversion were relatively the same in the absence and the presence of C₂H₄. Specifically, both conversion parameters showed less than a 30% relative change. The minor change in the conversion parameters may be linked to minor differences in the feed composition, reactor operating conditions, or both. From these observations it can be inferred that presence of ethylene in the feed stream does not affect the catalyst performance.

The results of Test ID G may also be compared to Test ID F to determine the effects of H₂ on the process as the operating conditions for the reactors were relatively similar. Specifically, the GHSV was less than 30% relative difference, the reaction temperature was the same, and the reactor in the pressure was nearly identical. However, in the Test ID G, ethane is present in the feed stream, while in Test ID F, ethane is absent from the feed stream. Further, Test ID F has a feed composition that includes H₂, while the feed composition of reactant in Test ID G does not include H₂.

Bearing in mind that above difference and similarities in the reactor operating condition and feed composition between the two examples, one can make the following observation. The CO₂ conversion and hydrocarbon conversion increased when H₂ was present in the feed stream. The increase in the conversion of the mentioned reactants was not attributed to presence or absence of feed ethane. This is because based on the comparison of results of Test ID C to Test ID F, it was inferred that feed mixture of ethane/CO₂/steam and feed mixture of ethylene/ethane/CO₂/steam show comparable conversion of the mentioned reactants when the reactor operating conditions are relatively similar. From this observation, it can infer that presence of hydrogen in C₂H₄—C₂H₆—CO₂—H₂O feed mixture stream would increase the CO₂ conversion and hydrocarbon conversion to value added products (mainly acetic acid).

TABLE 8

Reactor Feed Composition Ranges and Operating Condition Ranges

| Test ID | GHSV (h⁻¹) | WHSV (h⁻¹) | Reaction Temp. (°C) | Reaction Inlet Pressure (psig) | Feed Composition (Vol %) | | | |
|---|---|---|---|---|---|---|---|---|
| | | | | | H₂O | CO₂ | C₂H₆ | C₂H₄ |
| G | 612 | 1.01 | 320 | 13-16 | 30 | 30 | 15 | 25 |

TABLE 9

Catalyst activity and Product Distribution for Experiment Conducted in this Section

| Test ID | Reaction Temperature (°C) | Reaction Inlet Pressure (psig) | Hydrocarbon Conversion (C-atom %) | CO₂ feed Conversion (C-atom %) | Acetic Acid Selectivity (C-atom %) |
|---|---|---|---|---|---|
| G | 320 | 13-16 | 0.86 | 3.49 | 100 |

### Embodiments

An embodiment described herein provides a method for producing acetic acid in a selective oxidation (SO) reactor. The method includes providing a fresh feed stream to the SO reactor. The fresh feed stream includes a light hydrocarbon feed stream, a carbon dioxide feed stream, and a steam feed stream. Acetic acid is formed in the SO reactor. An acetic acid product stream is separated from a reactor effluent stream in a scrubber. A recycle gas stream is obtained from the scrubber, and at least a portion of the recycle gas stream is combined into the fresh feed stream to the SO reactor.

In an aspect, two or more feed streams are added separately to the SO reactor. In an aspect, an amount of the steam feed stream added to the SO reactor is adjusted to increase a selectivity for acetic acid. In an aspect, a flue gas stream from a combustion process is used as the carbon dioxide feed stream.

In an aspect, an ethane stream is used as the light hydrocarbon feed stream. In an aspect, a mixed stream including ethane and ethylene is used as the light hydrocarbon feed stream. In an aspect, a recycled water stream is separated from the acetic acid, and at least a portion of the recycled water stream is combined with the steam feed stream.

In an aspect, the fresh feed stream is placed in contact with an SO catalyst including a compound of formula:

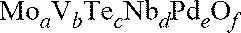

wherein a, b, c, d, e and f are relative atomic amounts of elements Mo, V, Te, Nb, Pd and O, respectively; and wherein when a is 1, b is between 0.01 and 1.0, c is between 0 and 1.0, d is between 0 and 1.0, e is between 0 and 0.10, and f is a number to at least satisfy a valence state of metallic elements in the SO catalyst. In an aspect, the fresh feed stream is placed in contact with an SO catalyst that includes vanadium.

In an aspect, the acetic acid product stream is produced in one reactor of a number of parallel SO reactors, and an ethylene product stream is produced in at least one other reactor of the number of parallel SO reactors. In an aspect, the acetic acid product stream from the one reactor of the number of parallel SO reactors is combined with a feed stream provided to the other reactor of the number of parallel SO reactors.

Another embodiment described herein provides a reactor system for producing acetic acid in a selective oxidation process. The reactor system includes a selective oxidation (SO) reactor. The SO reactor includes a number of feed lines including a light hydrocarbon feed line, a carbon dioxide feedline, and a steam feedline. The SO reactor includes an SO catalyst to convert feedstocks, at least in part, to acetic acid, and a reactor effluent line. The reactor system includes a scrubber coupled to the reactor effluent line. The scrubber includes an acetic acid product line and a separated gas outlet. The separated gas outlet is coupled to one of the number of feed lines to recycle at least a portion of a gas stream separated from an acetic acid product stream to the SO reactor.

In an aspect, at least two of the number of feed lines are combined to form a single feedline to the SO reactor.

In an aspect, a number of SO reactors are used, wherein each of the number of SO reactors is coupled to a dedicated scrubber. The dedicated scrubber for each SO reactor is configured to separate the reactor effluent into the acetic acid product stream in the gas stream. The dedicated scrubber is configured to either return the gas stream to an SO reactor coupled to the dedicated scrubber, or provide the gas streams to a subsequent SO reactor.

In an aspect, the acetic acid product stream from the scrubber is coupled to another SO reactor which is producing ethylene. In an aspect, the light hydrocarbon feed line is coupled to a recycle line from an ODH system to provide a mixture of ethane and ethylene to the SO reactor. In an aspect, the light hydrocarbon feed line is coupled to a C₂ splitter to provide ethane to the SO reactor. In an aspect, the carbon dioxide feedline is coupled to a carbon dioxide pipeline. In an aspect, the carbon dioxide feedline is coupled to a flue gas line from a combustion process. In an aspect, a process skid includes the SO reactor and the scrubber.

Another embodiment described herein provides a method for producing acetic acid in a selective oxygenation (SO) reactor. The method includes providing a fresh feed stream to the SO reactor. The fresh feed stream includes an ethane feed stream, a carbon dioxide feed stream provided from flue gas stream from a combustion process, and a steam feed stream. Acetic acid is formed in the SO reactor, wherein an amount of water added to the SO reactor as the steam feed stream is adjusted to increase selectivity for acetic acid. An acetic acid product stream is separated from a reactor effluent stream in a scrubber. A recycle ethane stream is obtained from the scrubber, and at least a portion of the recycle ethane stream is combined into the fresh feed stream to the SO reactor.

In an aspect, the fresh feed stream is placed in contact with an SO catalyst including a compound of formula:

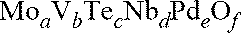

wherein a, b, c, d, e and f are relative atomic amounts of elements Mo, V, Te, Nb, Pd, and O, respectively; and wherein when a is 1, b is between 0.01 and 1.0, c is between 0 and 1.0, d is between 0 and 1.0, e is between 0 and 0.10, and f is a number to at least satisfy the valence state of metallic elements in the SO catalyst. In an aspect, the fresh feed stream is placed in contact with an SO catalyst that includes vanadium.

A number of implementations have been described. Nevertheless, it will be understood that various modifications may be made without departing from the spirit and scope of the disclosure.

### INDUSTRIAL APPLICABILITY

The present disclosure relates to a method and reactor system for the production of acetic acid by selective oxidation of a feed comprising a light hydrocarbon, carbon dioxide and steam.

1. A method for producing acetic acid in a selective oxidation (SO) reactor, comprising:
providing a fresh feed stream to the SO reactor, wherein the fresh feed stream comprises:
a light hydrocarbon feed stream;
a carbon dioxide feed stream; and
a steam feed stream; and
forming acetic acid in the SO reactor;
separating an acetic acid product stream from a reactor effluent stream in a scrubber;
obtaining a recycle gas stream from the scrubber; and
combining at least a portion of the recycle gas stream into the fresh feed stream to the SO reactor.

2. The method of claim 1, further comprising adding two or more feed streams separately to the SO reactor.

3. The method of claim 1, further comprising adjusting an amount of the steam feed stream added to the SO reactor to increase a selectivity for acetic acid.

4. The method of claim 1, further comprising using a flue gas stream from a combustion process as the carbon dioxide feed stream.

5. The method of claim 1, further comprising using an ethane stream as the light hydrocarbon feed stream.

6. The method of claim 1, further comprising using a mixed stream comprising ethane and ethylene as the light hydrocarbon feed stream.

7. The method of claim 1, further comprising:
separating a recycled water stream from the acetic acid; and
combining at least a portion of the recycled water stream with the steam feed stream.

8. The method of claim 1, further comprising placing the fresh feed stream in contact with an SO catalyst comprising a compound of formula: 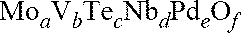
wherein a, b, c, d, e and f are relative atomic amounts of elements Mo, V, Te, Nb, Pd, and O, respectively; and wherein when a is 1, b is between 0.01 and 1.0, c is between 0 and 1.0, d is between 0 and 1.0, e is between 0 and 0.10, and f is a number to at least satisfy a valence state of metallic elements in the SO catalyst.

9. The method of claim 1, further comprising placing the fresh feed stream in contact with an SO catalyst that comprises vanadium.

10. The method of claim 1, wherein the recycle gas stream comprises ethane, ethylene, or a mixture thereof.

11. The method of claim 1, further comprising:
producing the acetic acid product stream in one reactor of a plurality of parallel SO reactors; and
producing an ethylene product stream in at least one other reactor of the plurality of parallel SO reactors.

12. The method of claim 11, further comprising combining the acetic acid product stream from the one reactor of the plurality of parallel SO reactors with a feed stream provided to the at least one other reactor of the plurality of parallel SO reactors.

13. A reactor system for producing acetic acid in a selective oxidation process, comprising:
a selective oxidation (SO) reactor, comprising:
a plurality of feed lines comprising:
a light hydrocarbon feed line;
a carbon dioxide feed line; and
a steam feed line;
an SO catalyst to convert feedstocks, at least in part, to acetic acid; and
a reactor effluent line; and
a scrubber coupled to the reactor effluent line, comprising:
an acetic acid product line; and
a separated gas outlet, wherein the separated gas outlet is coupled to one of the plurality of feed lines to recycle at least a portion of a gas stream separated from an acetic acid product stream to the SO reactor.

14. The reactor system of claim 13, wherein at least two of the plurality of feed lines are combined to form a single feed line to the SO reactor.

15. The reactor system of claim 13, further comprising a plurality of SO reactors, wherein each of the plurality of SO reactors is coupled to a dedicated scrubber, wherein the dedicated scrubber is configured to separate the reactor effluent into the acetic acid product stream and the gas stream, and wherein the dedicated scrubber is configured to either:
return the gas stream to an SO reactor coupled to the dedicated scrubber; or
provide the gas stream to a subsequent SO reactor.

16. The reactor system of claim 15, wherein the acetic acid product stream from the scrubber is coupled to another SO reactor which producing ethylene.

17. The reactor system of claim 13, wherein the light hydrocarbon feed line is coupled to a recycle line from an ODH system to provide a mixture of ethane and ethylene to the SO reactor.

18. The reactor system of claim 13, wherein the light hydrocarbon feed line is coupled to a C₂ splitter to provide ethane to the SO reactor.

19. The reactor system of claim 13, wherein the carbon dioxide feed line is coupled to a carbon dioxide pipeline.

20. The reactor system of claim 13, wherein the carbon dioxide feed line is coupled to a flue gas line from a combustion process.

21. The reactor system of claim 13, further comprising a process skid comprising the SO reactor and the scrubber.

22. A method for producing acetic acid in a selective oxygenation (SO) reactor, comprising:
providing a fresh feed stream to the SO reactor, wherein the fresh feed stream comprises:
an ethane feed stream;
a carbon dioxide feed stream provided from a flue gas stream from a combustion process; and
a steam feed stream; and
forming acetic acid in the SO reactor, wherein an amount of water added to the SO reactor as the steam feed stream is adjusted to increase selectivity for acetic acid;
separating an acetic acid product stream from a reactor effluent stream in a scrubber;
obtaining a recycle ethane stream from the scrubber; and
combining at least a portion of the recycle gas stream into the fresh feed stream to the SO reactor.

23. The method of claim 22, further comprising placing the fresh feed stream in contact with an SO catalyst comprising a compound of formula: 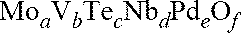
wherein a, b, c, d, e and f are relative atomic amounts of elements Mo, V, Te, Nb, Pd, and O, respectively; and wherein when a is 1, b is between 0.01 and 1.0, c is between 0 and 1.0, d is between 0 and 1.0, e is between 0 and 0.10, and f is a number to at least satisfy the valence state of metallic elements in the SO catalyst.

24. The method of claim 22, further comprising placing the fresh feed stream in contact with an SO catalyst that comprises vanadium.
